(19) United States
(12) Patent Application Publication
(10) Pub. No.: US 2023/0140615 A1
(43) Pub. Date: May 4, 2023

(54) MONITORING OF CARDIAC ACTIVITY

(71) Applicant: DREEM, Paris (FR)

(72) Inventors: Quentin SOULET DE BRUGIERE, Paris (FR); Martin HERRERA, Paris (FR); Manon SOUBRIER, Paris (FR)

(73) Assignee: DREEM, Paris (FR)

(21) Appl. No.: 17/917,773

(22) PCT Filed: Apr. 7, 2021
(86) PCT No.: PCT/FR2021/050605

§ 371 Date: Oct. 7, 2022

(30) Foreign Application Priority Data
Apr. 8, 2020 (FR) 20 03542

(51) Int. Cl. A61B 5/30; A61B 5/28; A61B 5/273; A61B 5/256; A61B 5/263; A61B 5/00

(52) U.S. Cl. CPC A61B 5/303; A61B 5/28; A61B 5/273; A61B 5/256; A61B 5/263; A61B 5/6803; A61B 5/681; A61B 2562/182; A61B 5/7203

(57) ABSTRACT

A heart activity measurement device for an individual including: a processing unit; at least one measurement cable, including a first end suited for connection to the processing unit in order to form an input for an electrical potential, and a second end; a support, ready for an individual to wear, where the support supports the cable so as to keep a predefined path for the cable relative to the skin of the individual when the support is worn. The cable is laid out such that when the support is worn, any contact between an electrically conducting part of the cable and the skin of the individual is prevented. The second end of the cable does not have any electrode for contact with the skin. The support is further laid out for keeping a predefined spacing between a measurement portion of the cable and the skin of the individual.

### FIELD

The present disclosure relates to the field of bioelectric signal measurement, and more specifically the measurement of heart activity. It also relates to a portable device for acquiring heart activity signals from an individual.

### BACKGROUND

During heartbeats, physiological electrical signals are generated by a specific category of heart cells. These electrical signals travel in the heart leading to the appearance of electrical potentials which are responsible for heart muscle activity.

The possibility of measuring these electrical potentials via an electrocardiograph in order to obtain an electrocardiogram (ECG) representing graphically the heart's electrical activity has been known for a long time.

Using the graphical data, it is then possible to evaluate the proper functioning of the heart edited tact cardiac anomalies (e.g. arrhythmia). With early detection of heart problems, monitoring can be established which may, for example, be coupled with an appropriate treatment (medication), or which may even require placing a heart device (e.g. pacemaker).

The devices with which to do ECG measurements have become smaller and smaller and less invasive. Some are portable, so the inconvenience and discomfort for an individual in their everyday life can be minimized and the degree of monitoring also increased.

All the same, these portable ECG devices still remain much too invasive in an individual's life, in particular when it involves adhering electrodes to the surface of the skin.

### SUMMARY

The present disclosure aims to improve that situation.

A heart activity measurement device for an individual is therefore proposed, where the device may comprise:

a processing unit;
at least one measurement cable, including a first end suited for connection to the processing unit in order to form an input for an electrical potential, and a second end;
a support, ready for an individual to wear, where said support supports the cable so as to keep a predefined path for the cable relative to the skin of the individual when the support is worn by the individual;
where the cable is further laid out such that when the support is worn by the individual, any contact between an electrically conducting part of the cable and the skin of the individual is prevented;
the second end of the cable does not have any electrode for contact with the skin;
The support is further laid out for, when the support is worn by the individual, keeping a predefined spacing (e) between a measurement portion of the cable and the skin of the individual, where the predefined spacing is included between 0.5 and 20 mm.

In that way advantageously, the preceding arrangements may allow a reduction of the bulk of a heart activity measurement device, but also a reduction of the inconvenience experienced by an individual, in particular because of the typical direct contact of an electrode.

This small bulk may allow an individual, because of a suitable support, wear the device on body parts which are less constraining for daily life, while also allowing good quality heart activity measurements. The device may be worn in the area of the forehead of the individual, a wrist to the individual or even near an ankle of the individual.

Support means the parts not directly having an electronic and measurement function but instead a mechanical function of keeping the electronic elements near/on/around the individual.

Path may be understood as the way in which a cable is arranged relative to the support and nearby or in electrical contact with the surface of the skin.

Near is understood to mean that the measurement cable can be close to the surface of the skin without necessarily being in physical or electrical contact therewith. Near may be defined according to the predefined spacing with the skin, and may be included between 0.2 and 40 mm. Preferably, a predefined spacing is included between 0.5 and 20 mm.

The length of the predefined path of the measurement cable may be included between 0.05 and 5 m. Preferably the length of the predefined path may be included between 0.15 and 3 m.

The measurement cable may be formed from a conducting wire made of a metal (e.g. copper, aluminum, gold, nickel, etc.), and surrounded by an insulating material (e.g. polymer) performing an unshielded measurement cable.

The insulating part may further be surrounded by a second conductor (electrically insulated from the first) in the form of a metal braid so as to form a shielding (i.e. a shielded measurement cable). The metal braid may comprise copper or aluminum. The total diameter of the cable may be included between 0.2 and 2 mm. The diameter of the wire of the conducting may be included between 0.09 and 1.8 mm. The thickness of the insulating or dielectric material surrounding the conducting wire may be included between 0.07 and 1.9 mm. Preferably, the total diameter of the cable may be included between 0.2 and 0.5 mm, the diameter of the conducting wire between 0.09 and 0.3 mm, and/or the thickness of the insulator between 0.07 and 0.1 mm.

In one or more embodiments, the device may further comprise:

at least one reference cable, including a first end suited for connection to the processing unit in order to form an electrical reference, or bias,
where the reference cable is supported by the support such that, when the support is worn by the individual:
an electrically conducting part of the reference cable is near or in electrical contact with the skin of the individual; and
a predefined path of the reference cable extends along the skin.

Electrical reference may be understood to be a different electrical reference from the bulk ground of the device. The electrical reference may be a voltage value different from 0 V, and may be measured near or in electrical contact with the skin. This reference cable connected to a voltage following device may serve to reduce the common mode voltage. The voltage value of the electrical reference may be included between −12 and 12 V, preferably included between −5 and 5 V, and more preferably included between 2 and 3 V.

The predefined length of the reference cable may be included between 0.05 m and 3 m. Preferably, the length of the predefined path may be included between 0.15 and 0.50 m.

According to one or more embodiments, the measurement cable may include a shield, thus forming a coaxial type cable, where said shield could be connected to the processing unit as bulk ground.

The central core (first conductor) of the cable corresponds to the conducting wire of a coaxial type cable and the metal braiding (second conductor) corresponding to the shielding of the coaxial type cable.

The use of a measurement cable with shielding, such as for example coaxial type cable, may allow measuring an electric potential via a double coaxial capacitor. The first capacitor may be formed between the skin of the individual and the shielding (i.e. the ground of the measurement cable), and the second capacitor may be formed between the shielding and the conducting wire of the coaxial type measurement cable.

Advantageously, the measurement of the heart activity of an individual by double coaxial capacitor effect may serve to get a good precision in the measurement, while keeping a minimal bulk of the device. In particular, it allows a measurement without necessarily having two points of contact, unlike the conventional ECG.

In one or more embodiments, the device may further comprise at least one additional measurement cable including a first end suitable for connection to the processing unit in order to form an input of at least one second electrical potential, where the support further supports the additional measurement cable so as to maintain a predefined path for the additional measurement cable relative to the skin of the individual when the support is worn by the individual.

Advantageously, the use of an additional measurement cable may serve to improve the sensitivity of the heart activity measurement of the person P in the presence of parasitic electrical noise. The use of an additional measurement cable may serve to improve the signal-to-noise ratio for electrical potential measurements. The result of this is a more precise determination of the heart activity of the person P.

By combining the signals, in particular comparison, information obtained after measurement data processing by the processing unit may be more precise and reliable for qualifying the heart activity of the individual.

The length of the predefined path of the additional measurement cable may be included between 0.05 and 3 m. Preferably the length of the predefined path may be included between 0.5 and 1.5 m.

The structure of the additional measurement cable may be the same kind as the structure of the measurement cable, or may be different. The additional measurement cable may be a shielded or coaxial type shielded cable, or even an unshielded cable like a conducting wire surrounded by an insulating material or not. The use of a shielded structure for the additional measurement cable may have the advantage of improving the signal-to-noise ratio.

According to another aspect, the device may further comprise at least one surface electrode supported by the support such that when the support is worn by the individual, each of the at least one electrode is in electrical contact with the skin of the individual,

where the at least one cable of the device other than the measurement cable can be connected to at least one electrode for placement in electrical contact with the skin of the individual via said electrode.

The use of at least one surface electrode with at least one cable for the device other than the measurement cable can serve to improve a little more the measurement sensitivity of the electric potentials corresponding to the heart activity of the individual. More precisely, the use of the surface electrode may serve to improve a little more the signal-to-noise ratio of the one or more measurements.

In that way, it is possible to increase the precision of the information obtained from the measurements processed by the processing unit.

In one or more embodiments, the at least one electrode may be a dry electrode, making the addition of a contact fluid unnecessary.

In one or more embodiments, the at least one electrode may comprise carbon doped silicon.

In one or more embodiments, the support may be shaped as:

an article of headwear which can be worn on or around the head of an individual, or
a bracelet which can be worn on the arm or wrist of an individual.

In one or more embodiments, the predefined path of the measurement cable may have a length greater than or equal to 15 cm.

### BRIEF DESCRIPTION OF THE DRAWINGS

The characteristics disclosed in the following paragraphs, may optionally be implemented. They may be implemented independently of each other or in combination with each other. Other characteristics, details and advantages will appear upon reading the following detailed description, and upon analysis of the attached drawings, in which:

FIG. 1 shows an example of a heart activity measurement device for an individual in one or more embodiments.

FIG. 2a shows an illustration, in one or more embodiments, of a predefined path of at least one measurement cable included in the device worn by an individual P.

FIG. 2b shows a section in the (y, z) plane of the measurement device shown in FIG. 2a.

FIG. 2c shows, in one or more embodiments, an alternative predefined path of the measurement cable described in FIG. 2a.

FIG. 3a is an illustration, in one or more embodiments, of predefined paths of at least one measurement cable and several additional cables included in the device worn by an individual P.

FIG. 3b shows an example of a layout of the cables of the device from FIG. 3a according to a (y, z) section similar to that from FIG. 2b.

FIG. 3c shows, in one or more embodiments, an alternative to the predefined path of the cables described in FIG. 3a.

FIG. 4 shows, in one or more embodiments, a configuration example of a heart activity measurement device worn near the wrist of the individual P.

FIG. 5 shows, in one or more embodiments, a configuration example of a heart activity measurement device worn near the wrist of the individual P.

FIG. 6 shows a schematic representation of a heart activity measurement device for an individual in one or more embodiments.

### DETAILED DESCRIPTION

The following drawings and description contain, for the most part, elements of a definite kind. They could therefore not only serve to better understand the present disclosure, but also contribute to definition thereof, as applicable.

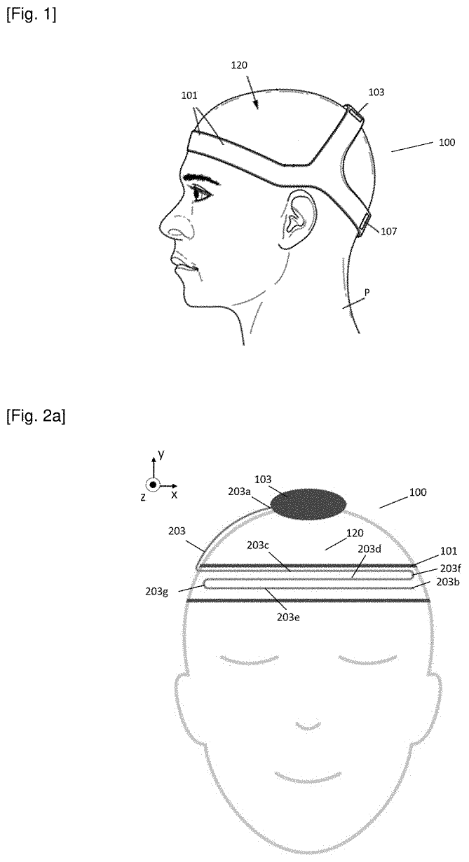

FIG. 1 shows an example of a heart activity measurement device for an individual in one or more embodiments.

A heart activity measurement device 100 comprises a support 101 suited for being worn by a person P, whether for a period of activity (e.g. sports) for example, or of inactivity (e.g. sleep). The support 101 (e.g. in the form of a band) is, here, suited for being worn around the head of the person P. For this purpose, the support 101 is suited for surrounding the head of the person P at least partially and so as to be kept in place thereon. The support 101 is for example suited for surrounding at least a part of the circumference of the head 120 (for example near the surface of the skin of the forehead) of the person P, or at least half of a circumference of the head of the person P. In the embodiment shown here, the support 101 is suited for fully surrounding the head of the person P.

As a variant, the support is suited for being worn around a limb of the individual P, such as for example near the wrist (e.g. in the form of a bracelet).

The device 100 further comprises a processing unit 103 and a battery 107, supported by the support 101. The processing unit 103 may be connected to one or more cables supported by the support 101. The processing unit 103 is further configured for collecting information, in particular through electrical signals received and transported by the cables.

According to an example, the cables may be incorporated inside the support 101, meaning that the cables are neither visible nor accessible from outside the support 101. When the cables are located inside the support 101, they can preferably be located closest to the inner surface of the support 101 and therefore closest to the individual P (i.e. to the surface of the skin of the individual P).

As a variant, the cables are located on the outer surfaces of the support, and preferably on the outer surface of the part of the support 101 in contact with the forehand (i.e. the surface of the skin) of the person P.

Each cable of the device 100 comprises a conducting wire, which is optionally surrounded by insulating material such as a polymer, thus forming a cable referred to as unshielded.

As an example, according to the “AWG Gauge” standard, the unshielded cable may be a reference cable between AWG #44 and AWG #15 defining a cable whose conducting section is equivalent to that of a diameter ranging from 0.05 mm to 1.45 mm (thus covering the case of a multistrand conducting wire whose apparent diameter is greater than that of the conducting section).

According to an alternative, for one or more cables for the device 100, the insulating material comprises at least one dielectric material (e.g. polyethylene (PE), polypropylene (PP), fluorinated ethylene propylene (FEP) and polytetrafluoroethylene (PTFE)). The dielectric material may be surrounded with a second conductor (e.g. in metal braid form) thus forming a shielded cable. According to an example, the shielded cable may be a coaxial cable.

According to the AWG gauge standard, the shielded cable may, for example, be in AWG #40 reference cable defining a cable for which the conducting section of the conducting wire is equivalent to that of a conducting cylinder with a diameter of about 0.08 mm.

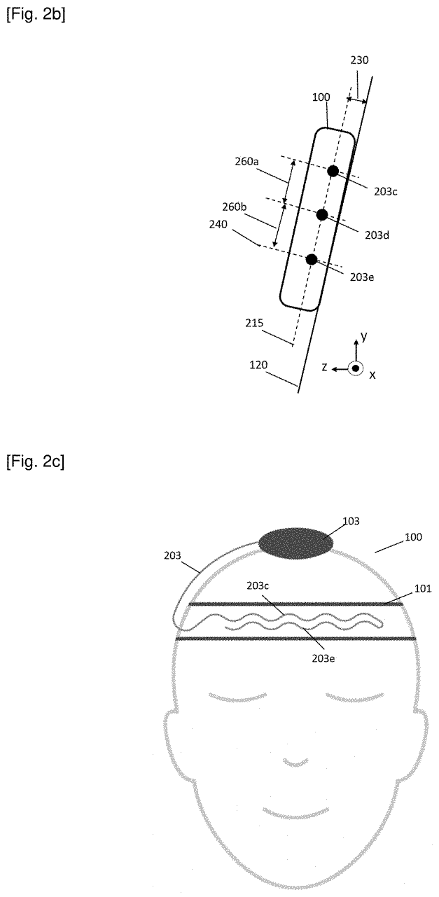

FIG. 2a shows an illustration, in one or more embodiments, of a predefined path of at least one measurement cable included in the device 100 worn by an individual P.

FIG. 2a may correspond to a front view of the person P (or individual P). The support 101 of the device 100 is shown from the front and transparent so that the measurement cable 203 can be seen.

In that way, the measurement cable 203, here shielded (e.g. coaxial), may be connected via the first end 203a thereof to the processing unit 103 included in the heart activity measurement device 100, while the shielding of the measurement cable 203 is connected to the bulk ground of the processing unit 103. The second end 203b of the cable 203 may be located at the end of a predefined path, and be left free, meaning not connected to a third element. In particular, the second end 203b remains disconnected from any electrode in electrical contact with the skin.

Because of the predefined path, the measurement cable 203 may serve to measure a first electrical potential (or a vale of a first electrical potential) by a double coaxial capacitor effect. The electrical potential may be an electrical potential generated during the heart cycle of the individual P, and propagating to the surface of the body.

In fact, a first capacitor may be defined between the skin of the person P and the shielding (conducting braid or sheet forming the bulk ground) of the measurement cable 203. A second coaxial capacitor may be defined between the cable core (coaxial), and the shielding.

In the example shown in FIG. 2a, the predefined path corresponds to the continuation of straight portions 203c; 203d; 203e and curved portions 203f; 203g. “Straight” is understood here as distinguished from the “curved” portions. Just the same, it is understood that “straight” portions themselves have a small curvature corresponding to that of a human forehead. Here, the path has a general sinusoidal or “serpentine” shape. The straight portions extend substantially along the x axis corresponding to the width of the forehead 120 of the individual P.

According to an alternative, the straight portions may extend along directions of the height of the forehead of the individual (i.e. along the y axis FIG. 2a).

The curved portions may be, for example, in a semicircle shape, or in a semi-ellipse shape.

FIG. 2b shows a section in the (y, z) plane of the measurement device 100 shown in FIG. 2a.

The section view thus shows a possible arrangement when the predefined path (e.g. straight portion along x axis) of the measurement cable 203 is housed within the support 101, where the support 101 is in contact with the forehead 120 of the person P.

For example, each portion of the cable 203c; 203d; 203e can be separated from the skin (here of the forehead 120) by a predefined spacing 230. Conventionally, the predefined separation 230 is measured between the center of each portion of cable and the surface (the skin) of the forehead 120 of the person P. In FIG. 2b, the reference 215 represents a surface passing by the center of the cable all along the path thereof. In a situation in which the predefined spacing 230 is uniform over the entire path of the cable 203, the surface 215 is therefore located the same distance from the forehead 120 of the individual P at every point.

As a variant, the predefined separation 230 may vary along the cable 203, in such a way that some portions are closer to the surface of the skin than others, even in electrical contact.

Thus, according to an embodiment, the predefined path of the measurement cable 203 may be arranged in such a way that the cable does not come into contact with the surface of the forehead 120 of the individual P.

According to an example, this predefined spacing 230 may be included between 0.1 and 30 mm, whether it is substantially uniform or not. Preferably, this predefined spacing 230 may be included between 0.5 and 20 mm. Also preferred, this predefined spacing 230 may be included between 0.4 and 2.5 mm.

In the example described here, the predefined path of the cable 203 is arranged such that the portions of the measurement cable 203 do not cross above the forehead 120. In particular any contact between two portions of the measurement cable is avoided.

Each portion of cable can be separated from one or more neighboring portions by a predefined distance. For example, this separation may be measured between the centers 240 of each portion of cable.

As an example, the portion of cable 230d is separated from the portion of cable 230c by a separation distance 260a and is separated from the portion 203e by a separation distance 260b.

According to an example, the separation distance between each portion of the measurement cable 203 is included between 0 (contact) and 20 mm. Preferably it is included between 0 and 10 mm.

According to the alternative, the separation distance may be identical for each separation, or identical for some separation, or even be different for each separation.

In that way, the predefined path may advantageously be arranged so as to effectively optimize the measurement of one or more electrical potentials at the surface of the body (e.g. the surface of the forehead of the person P) while also keeping a minimal bulk of the measurement means, and without limitation on the individual.

FIG. 2c shows, in one or more embodiments, an alternative predefined path of the measurement cable described and shown in FIG. 2a.

In this alternative, the characteristics of the device 100 shown in FIG. 2a and FIG. 2b, in particular, the characteristics and constraints relating to the position between the portions of cable, or the characteristics and constraints relating to the positioning relative to the support 101 and the individual P, are partially or completely transposable to the embodiment shown in FIG. 2c.

In this embodiment, the straight portions (e.g. 203c and 203e) of the predefined path of the measurement cable 203 shown in FIG. 2a may have the shape of a sinusoidal configuration instead of the shape of a straight configuration.

The advantage of such a shape in the predefined path may be optimization of the bulk of the measurement cable. Another advantage may be guaranteeing the integrity of the measurement cable. In fact since the support 101 is slightly elastic, the elongation of the support worn by an individual can lead to tensional stress on straight portions of the cable. Such stresses may in time lead to damage of the measurement cable.

According to another alternative, the predefined path may have the shape of any configuration suited for limiting the bulk of the measurement cable, while also keeping the significant total length and therefore a reliable electrical interaction.

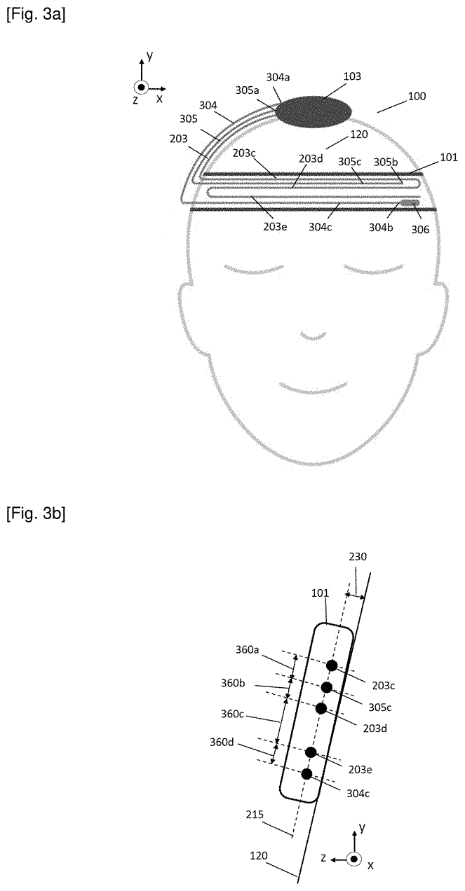

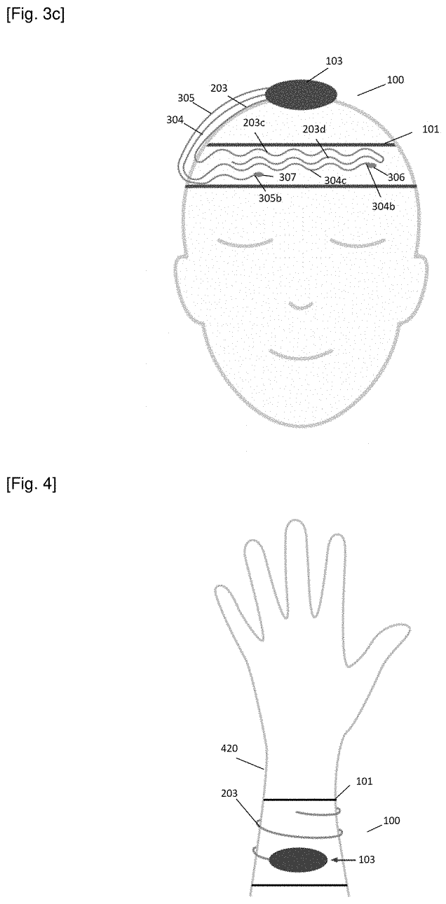

FIGS. 3a and FIG. 3b show an alternative to the embodiments shown in FIG. 2a and FIG. 2b.

In this alternative, the characteristics of the device 100 shown in FIG. 2a,

FIG. 2b and FIG. 2c, in particular, the characteristics and constraints relating to the position between the portions of cable, and also the characteristics and constraints relating to the positioning relative to the support 101 and the individual P, are partially or completely transposable to the embodiment shown in FIG. 3a and FIG. 3b.

FIG. 3a is an illustration, in one or more embodiments, of predefined paths of at least one measurement cable and several additional cables included in the device worn by an individual P.

In comparison with the previous embodiment, the measurement cable 203 is unshielded. As a variant, the measurement cable 203 may be shielded.

Additional cables may have different respective functions.

For example, one of the additional cables for the device 100 may be an additional measurement cable 304. Analogously to the measurement cable 203, the additional measurement cable 304 is supported by the support 101 and may have similar structural characteristics to those described above relating to the measurement cable 203.

According to a variant, the measurement cable 203 and the supplemental measurement cable 304 may have different structural characteristics.

The additional measurement cable 304 comprises two ends 304a; 304b. The first end 304a may be connected to the processing unit 103. The second end 304b may be connected to a third element such as an electrode 306 intended to come into contact with the skin. A dry and/or carbon doped electrode is particularly reliable while also being less bothersome for the individual.

In the embodiment shown in FIG. 3a, the additional measurement cable 304 comprises a straight portion 304c extending along the predefined path of the measurement cable 203.

Thus, by means of the predefined path of the measurement cable 203 and the additional measurement cable 304, it is possible to measure respectively a first electrical potential and a second electrical potential relating to the heart activity of the individual P. The first electrical potential is obtained through a first capacitor defined between the skin of the person P and the conducting wire of the measurement cable 203. The second electrical potential is obtained through a second capacitor defined between the skin of the person P and the conducting wire of the supplemental measurement cable 304.

Thus, this embodiment, by using one or more measurement cables and also a reference cable, may allow increasing the signal-to-noise ratio and also the time resolution of the measured signals. A more precise measurement of the heart activity results.

In the example described here, one of the additional cables included in the device 100 may be a reference cable 305. This reference cable (or bias cable) supported by the support 101 may be used as an electrical reference. The reference cable 305 may have structural characteristics similar to those described above for the other cables.

In a variant, the reference cable 305 may have structural characteristics different from other cables of the device 100.

Further, analogously to the other cables of device 100, the reference cable 305 is connected to the processing unit 103 via a first end 305a. A second end 305b of the cable 305 is located at the end of a predefined path, and is left free, meaning not connected to a third element. The reference cable 305 extends, here, along the portion of the predefined path of the measurement cable 203. The reference cable 305 is positioned either near, or in electrical contact with the surface of the skin of the individual P.

The reference cable 305 serves to acquire a reference voltage value which can be used by the processing unit 103 for reducing the common mode voltage (CMV). In fact, the common mode voltage may correspond to a common voltage value on each measurement input, meaning a voltage value present both in the measurement of the first electric potential via the measurement cable 203 and in the measurement of the second electric potential via the additional measurement cable 304. When it is not distinguished from other voltage sources, the common mode voltage value may be problematic during measurement of low amplitude electrical signals. It may, for example, impact the precision of the measurement, even obscure the measurement sought.

In the present description, this common mode value may be included as the average value of the potential of the body of the individual P. This average potential value of the body may be subtracted by the processing unit 103 from the respective potential value measured at each cable.

According to a variant, the second end 305b of the reference cable 305 may be connected to a third element in electrical contact with the surface of the skin of the individual P. As an example, the third element (not shown in FIG. 3b) may be an electrode (e.g. dry and/or carbon doped electrode).

The use of an electrode may, for example, allow more precisely measuring the common mode voltage; and more reliably over time and in case of movement to the individual, and therefore improve the reduction of the CMV.

FIG. 3b shows an example of a layout of the cables of the device from FIG. 3a according to (i.e. (y, z)) section similar to that from FIG. 2b.

As described above, the characteristics and constraints relating to the position between the portions of cable or between the cables, and also the characteristics and constraints relating to the positioning relative to the support 101 and the individual P, are partially or completely transposable to the embodiment shown in FIG. 3b. For example, the similar constraints may be an arrangement of the cables without crossing of the respective portions thereof, of portions not brought into contact with the surface of the skin, or brought into contact with the surface of the skin.

Thus, as an example, each portion of the various cables 203c; 203d; 203e; 304c; 305c is spaced according to the predefined separation 230.

As a variant, respective predefined spacings for each cable (e.g. portion) may be arranged in such a way that some portions of different cables are closer than others to the surface of the skin, or even in electrical contact.

By comparison with the embodiment from FIG. 2b, the portion 305c of the reference cable 305 is separated from the portion 203c of the measurement cable 203 by a separation distance 360a, and is separated from the portion 203d of the measurement cable 203 by the separation distance 360b. Further, the portion 203c of the measurement cable 203 is separated from the portion 203d of the measurement cable 203 by a separation distance 360c, and is separated from the portion 304c of the additional measurement cable 304 by a separation distance 360d.

As previously described, as a variant, the separation distance may be identical for each separation, or identical for some separation, or even be different for each separation.

FIG. 3c shows, in one or more embodiments, an alternative to the predefined path of the cables described in FIG. 3a.

In this alternative, the characteristics of the device 100 shown in FIG. 3a and FIG. 3b, in particular, the characteristics and constraints relating to the position between the portions of cable or between the cables, as well as the characteristics and constraints relating to the positioning relative to the support 101 and the individual P, are partially or completely transposable to the embodiment shown in FIG. 2c.

In this embodiment, analogously to the embodiment shown in FIG. 2c, the straight portions of predefined path of the first measurement cable 203, of the additional measurement cable 304, and the reference cable 305 shown in FIG. 3a may have the shape of a sinusoidal configuration or any other shape suited for limiting the bulk of the various cables of the device.

According to an alternative, the respective ends 304b and 305b of each cable may be connected to an electrode 306; 307.

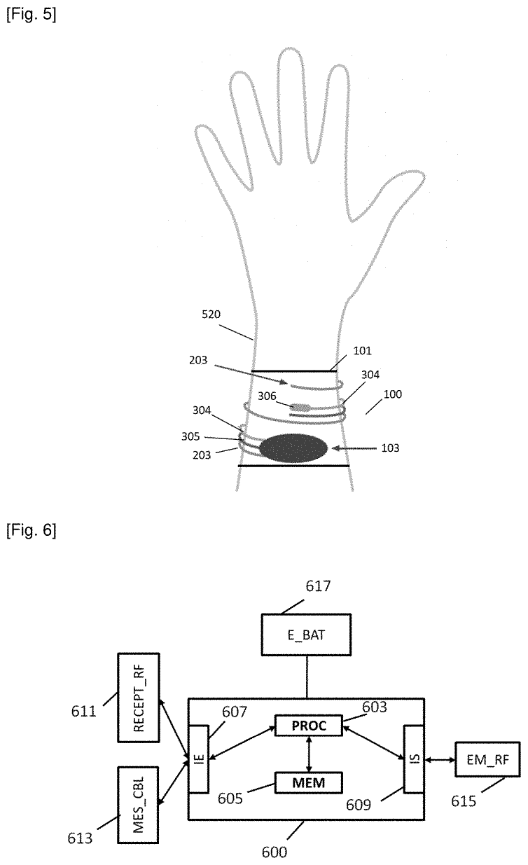

FIGS. 4 and 5 each show, in one or more embodiments, a sample configuration of a heart activity measurement device worn near the wrist 420; 520 of the individual P. The description relating to the embodiments from FIGS. 2a, 2b and 2c can be easily transposed, partially or completely, to the embodiments from FIG. 4 (single shielded cable), while the description relating to the embodiments from FIGS. 3a, 3b and 3c can be easily transposed to the embodiments from FIG. 5 (three cables). Generally, elements having the same numeric reference as elements from previous embodiments have the same functions, similar characteristics and the same possible variants.

Configurations of the device 100 worn at the wrist (or ankle) may therefore comprise similar elements to the configuration of the device worn on the head, and presented in the previous figures, including in terms of dimension.

In the embodiments suited to wearing at the wrist or ankle, the cable paths here take the shape of winding in spirals, preferably with several coils around the limb and without crossing.

One of the advantages of this embodiment (i.e. measurement device at the wrist or the ankle) may be to allow a discrete measurement of the heart activity in the daily life of an individual, for example at work, during sporting activity, etc.

FIG. 6 functionally shows a heart activity measurement device for an individual in one or more embodiments. Thus, the measurement device 600 comprises a memory 605 for storing instructions of a program. The memory 605 may further store measured data such as electric potential values. For example, this allows the device to at least temporarily be autonomous, for example over one night. In that way, all communication with third-party equipment is superfluous and the use of wireless communication means in the immediate proximity of a user over a long time is avoided.

The program may be executed by a processing circuit (or processing unit) 603 configured, at least, for measurement acquisition, such as, for example, electrical signals.

The processing circuit 603 may for example be:

a processor or processing unit suited for interpreting computer language instructions, where the processor or processing unit may comprise, or may be associated with, memory comprising instructions, or
the combination of a processor/processing unit and memory, where the processor or processing unit may be suited for interpreting computer language instructions with the memory comprising said instructions, or
a programmable electronic chip such as an FPGA chair (“Field Programmable Gate Array”).

The heart activity measurement device for an individual 600 further comprises an input interface 607 intended to be connected to cables (here globally designated by the reference 613) for measuring the electrical potentials produced during heart activity of an individual P or for determining an electrical reference. The input interface may also acquire data from telecommunications means 611, such as for example a radiofrequency receiver. Data acquisition may for example correspond to software and/or hardware updates of the measurement device 600.

The heart activity measurement device 600 further comprises an output interface 609 for sending information data about the heart activity to an individual by telecommunication means 615, like for example a radiofrequency transmitter. Information data about heart activity of an individual may for example be sent to a remote server or to a third device (e.g. telephone).

The device further comprises a battery 617 so as to supply energy to the various material components of the device 600.

The present description is not limited to the implementation examples described and represented above, from which other modes and other embodiments can be anticipated without going outside the scope of the present description.

1-9. (canceled)

10. A heart activity measurement device for an individual, said device comprising:
a processing unit;
at least one measurement cable, including a first end suited for connection to the processing unit in order to form an input for an electrical potential, and a second end;
a support, ready for an individual to wear, where said support supports the cable so as to keep a predefined path for the cable relative to the skin of the individual when the support is worn by the individual;
where the cable is further laid out such that when the support is worn by the individual, any contact between an electrically conducting part of the cable and the skin of the individual is prevented;
the second end of the cable does not have any electrode for contact with the skin;
the support is further laid out for, when the support is worn by the individual, keeping a predefined spacing (e) between a measurement portion of the cable and the skin of the individual, where the predefined spacing (e) is included between 0.5 and 20 mm

11. The device according to claim 10, further comprising:
at least one reference cable, including a first end suited for connection to the processing unit in order to form an electrical reference, or bias,
where the reference cable is supported by the support such that when the support is worn by the individual:
an electrically conducting part of the reference cable is near or in electrical contact with the skin of the individual; and
a predefined path of the reference cable extends along the skin.

12. The device according to claim 10, wherein, the measurement cable includes a shield, thus forming a coaxial type cable, where said shield could be connected to the processing unit as bulk ground.

13. The device according to claim 10, further comprising at least one additional measurement cable including a first end suitable for connection to the processing unit in order to form an input of at least one second electrical potential,
where the support further supports the additional measurement cable so as to maintain a predefined path for the additional measurement cable relative to the skin of the individual when the support is worn by the individual.

14. The device according to claim 10, further comprising at least one surface electrode supported by the support such that when the support is worn by the individual, each of the at least one electrode is in electrical contact with the skin of the individual,
where the at least one cable of the device other than the measurement cable can be connected to at least one electrode for placement in electrical contact with the skin of the individual via said electrode.

15. The device according to claim 10, wherein the at least one electrode is a dry electrode, making the addition of a contact fluid unnecessary.

16. The device according to claim 15, wherein the at least one electrode comprises carbon doped silicon.

17. The device according to claim 10, wherein the support is shaped as:
an article of headwear which can be worn on or around the head of an individual, or
a bracelet which can be worn on the arm or wrist of an individual.

18. The device according to claim 10, wherein the predefined path of the measurement cable may have a length greater than or equal to 15 cm.
